(19) United States
(12) Patent Application Publication
(10) Pub. No.: US 2019/0121803 A1
(43) Pub. Date: Apr. 25, 2019

(54) SCORING OF MICROMODULES IN A HEALTH PROGRAM FEED

(71) Applicant: KONINKLIJKE PHILIPS N.V., EINDHOVEN (NL)

(72) Inventors: SILVIA BERTAGNA DE MARCHI, WUERSELEN (DE); AKI SAKARI HARMA, EINDHOVEN (NL)

(21) Appl. No.: 16/142,588

(22) Filed: Sep. 26, 2018

Related U.S. Application Data
(60) Provisional application No. 62/565,395, filed on Sep. 29, 2017.

(51) Int. Cl. G06F 16/248; G06F 16/25; G06F 16/23; G06F 16/2457; G16H 20/30

(52) U.S. Cl. CPC G06F 16/248; G06F 16/252; G16H 20/30; G06F 16/24575; G06F 16/2379

(57) ABSTRACT

In an embodiment, a computer-implemented method for managing plural micromodules over time, each micromodule comprising plural nodes corresponding to electronic interventions that follow logically from one another to form a narrative or story, the method comprising: receiving inputs from one or more input sources, the input sources monitoring user behavior and associated contexts; monitoring each of the plural micromodules, each of the plural micromodules beginning with an opportunity and ending with an assessment, each of the plural micromodules comprising a score; updating the scores based on the received inputs; selecting which of at least one of the plural micromodules to provide to a user at any given moment in time based on a comparison of the scores and historical data; and providing the selected micromodule to the user.

### CROSS-REFERENCE TO RELATED APPLICATIONS

This patent application claims the priority benefit under 35 U.S.C. § 119(e) of U.S. Provisional Application No. 62/565,395 filed on Sep. 29, 2017, the contents of which are herein incorporated by reference.

### FIELD OF THE INVENTION

The present invention is generally related to health tracking based on wearable sensors and/or applications and related health self-management services.

### BACKGROUND OF THE INVENTION

Digital health programs typically are built based on static data structures or modules and sequential rules that govern the execution of the programs. Such modules may comprise electronic interventions (e.g., messaging or notifications) that are designed to influence a user towards healthy behavioral changes or lifestyle. A static module structure does not necessarily fit well with the lifestyle of an individual user and/or the dynamic changes in life situations and/or the environment around the user. A poor lifestyle-fit may lead to a low adherence, and hence effectiveness, of the digital health program. As an alternative to a fixed program, digital health programs may be designed that enable a user to stop a given module and possibly start it again later, but this does not remove the underlying problem that the module is not adapted to the user.

### SUMMARY OF THE INVENTION

In one embodiment, a computer-implemented method for managing plural micromodules over time, each micromodule comprising plural nodes corresponding to electronic interventions that follow logically from one another to form a narrative or story, the method comprising: receiving inputs from one or more input sources, the input sources monitoring user behavior and associated contexts; monitoring each of the plural micromodules, each of the plural micromodules beginning with an opportunity and ending with an assessment, each of the plural micromodules comprising a score; updating the scores based on the received inputs; selecting which of at least one of the plural micromodules to provide to a user at any given moment in time based on a comparison of the scores and historical data; and providing the selected micromodule to the user.

These and other aspects of the invention will be apparent from and elucidated with reference to the embodiment(s) described hereinafter.

### BRIEF DESCRIPTION OF THE DRAWINGS

Many aspects of the invention can be better understood with reference to the following drawings, which are diagrammatic. The components in the drawings are not necessarily to scale, emphasis instead being placed upon clearly illustrating the principles of the present invention. Moreover, in the drawings, like reference numerals designate corresponding parts throughout the several views.

FIG. 1 is a schematic diagram that illustrates an example environment in which a micromodule management system is used in accordance with an embodiment of the invention.

FIG. 2 is a schematic diagram that illustrates an example wearable device in which all or a portion of the functionality of a micromodule management system may be implemented, in accordance with an embodiment of the invention.

FIG. 3 is a schematic diagram that illustrates an example electronics device in which all or a portion of the functionality of a micromodule management system may be implemented, in accordance with an embodiment of the invention.

FIG. 4 is a schematic diagram that illustrates an example computing system in which at least a portion of the functionality of a micromodule management system may be implemented, in accordance with an embodiment of the invention.

FIG. 5 is a schematic diagram that conceptually illustrates an open-ended service environment of a health program that is managed by a micromodule management system, in accordance with an embodiment of the invention.

FIG. 6 is a schematic diagram that illustrates an example statement table used in conjunction with a micromodule management system, in accordance with an embodiment of the invention.

FIG. 7 is a schematic diagram that illustrates an example of candidate chains of electronic interventions used by a micromodule management system, in accordance with an embodiment of the invention.

FIG. 8 is a schematic diagram that illustrates an example of a history of micromodules monitored by a micromodule management system, in accordance with an embodiment of the invention.

FIG. 9 is a schematic diagram that illustrates an example of weighting of the micromodules as performed by a micromodule management system, in accordance with an embodiment of the invention.

FIG. 10 is a flow diagram that illustrates an example a micromodule management method, in accordance with an embodiment of the invention.

### DETAILED DESCRIPTION OF EMBODIMENTS

Disclosed herein are certain embodiments of a micromodule management system, method, and computer readable medium (herein, also collectively referred to as a micromodule management system) that increases the persuasiveness of health program content by managing micromodules (each of which includes a chain of insights) based on module history and a human model to enhance the adaptiveness of the health program.

Digressing briefly, and as indicated above, the current digital health program approach uses static, somewhat illustratively-described as brick wall, modules to govern execution of the health program. For instance, one week may utilize an activity module, followed on the next week with the activity module, then a nutrition module the next week, followed again by an activity module. These modules hence are designed under the assumption of a static lifestyle and/or environment. As an alternative to brick wall-type health programs, certain embodiments of a micromodule management system provide for an open-ended service that is built around the lifestyle of the user and which dynamically takes into account changes in the environment and the behavior of the user.

The micromodules comprise chains of electronic interventions (equivalently, statements, messages, notifications, all used interchangeably herein) that collectively provide a logical narrative or story over time. Each of the micromodules comprises a collection of nodes or data structures that are arranged according to a plurality of possible different paths or chains. Measures (e.g., scores) for the micromodules are computed based on statistics computed over time for each of the micromodules. Measures that meet a predefined criteria (e.g., greater than other scores and/or greater or equal to a predetermined threshold) are used to select the next node, which may be for the same micromodule or a different micromodule. Note that selection criteria for the next node may include the above and one or more additional criteria, including personalized criteria, features that meet historical criteria, or based on context aware features. As a statement is selected from among plural statements listed for a given node statement table, the statement is provided (e.g., published to a user), input data is received over an interval of time, and measures are computed for the various micromodules to select a next node statement for publication as a notification, and so on until a target node is reached. In the meantime, each notification (e.g., the issued statement) that is presented to the user is a logical step towards influencing a user in partaking in activities that improve the likelihood that the user will reach his or her goal. When the collection of notifications, though published in temporally different intervals, are viewed as a whole, the resulting chain of notifications that make up a micromodule comprises a narrative. That is, the statements or notifications comprise a logical relationship and provide a user-perceivable trend or direction towards a goal. The ongoing narrative provides continual, logical feedback and/or encouragement in assisting and/or advising the user in advancing progress towards a given goal. Certain embodiments of a micromodule management system orchestrates the issuance of the notifications according to an open-ended service based on data from one or more input sources (e.g., wearable devices comprising sensors, electronics devices, network storage (e.g., databases), etc.) and historical data.

Having summarized certain features of a micromodule management system of the present disclosure, reference will now be made in detail to the description of a micromodule management system as illustrated in the drawings. While a micromodule management system will be described in connection with these drawings, there is no intent to limit notification systems to the embodiment or embodiments disclosed herein. For instance, though described in the context of health management services, certain embodiments of a micromodule management system may be used to improve engagement of a user in other contexts, including financial management, business management, and industrial control processing. Note that the phrase, health management, refers to a broad set of services including fitness, health, and well-being, and includes not only clinical healthcare, but also personal healthcare, including baby care, baby development/monitoring, oral hygiene, skin care, shaving, etc., in addition to coaching, fitness and/or sports training, etc. Further, although the description identifies or describes specifics of one or more embodiments, such specifics are not necessarily part of every embodiment, nor are all various stated advantages necessarily associated with a single embodiment or all embodiments. On the contrary, the intent is to cover all alternatives, modifications and equivalents included within the spirit and scope of the disclosure as defined by the appended claims. Further, it should be appreciated in the context of the present disclosure that the claims are not necessarily limited to the particular embodiments set out in the description.

Note that throughout the specification, electronic interventions, statements, insights, and notifications are used interchangeably, the use of the terms notification or electronic intervention herein generally signifying that the statement is presented (published) electronically to a user via a device. In one embodiment, notifications may comprise a statement, wherein the statement comprises a reference to user data and a behavioral goal of the user and optionally a user preference. Statements are in the form of data structures, and according to the present disclosure are configured to convey information related to a plausible observation directed to the behavior of the user. The statements according to the present disclosure may be further configured to convey health-related information of the user. The statements may be presented as a fact that the user already recognizes. Further, the statements may be presented as a revealing of a hidden behavior pattern with advice to the user to change behavior to a better direction (e.g., improve health). In some embodiments, the electronic interventions may trigger circuitry of another device to perform a function or alter function, including alarm circuitry (e.g., for initiating visual, audible, haptic feedback), prosthetic or equipment control circuitry (e.g., triggering faster or slower treadmill speeds, resistance changes, such as to weight resistance equipment), among others. Certain embodiments of a micromodule management system automatically generates a large number of statements that are meaningful in a particular program context and selects nodes to link to a preceding node from among plural possible predefined pathways, wherein when viewed from the collective outcome of the various published statements over a period of time, the resulting chain or micromodule provides insight to the user (e.g., as presented at a user device) based on the node arrangement in narrative form. Measures or scores (those two terms likewise used herein interchangeably, the scores generally a subset of measures) are used in the initial defining of the statements, the selection of a starting node, and in the selection of subsequent nodes of the chain or micromodule to form a narrative to be presented to the user. In one embodiment, an assessment of a score for each of the micromodules is performed regularly, with the assessment enabling a decision on the next node (and hence statement) to present. In one embodiment, the measures or scores are computed for each micromodule, the scores based on statistical and heuristic weighting rules, and statements are selected for micromodules that have high scores (and meet a threshold value) in reaching a particular goal.

The digital health program according to the present disclosure is an application designed to be implemented on a mobile device (e.g., user device) to monitor physiological or psychological signs of the user as well as to track the real-time activities of the user. The program may be a health-related program or a health-related application. The programs developed for those devices employ one or more recommender-type systems to analyze the profile of the user and the historical data associated with one or more micromodules, and provide various types of messages to the user, or recommend one or more resources to the user. The statements individually comprise one or more personalized insights of the health-related behavior of the user. The statements may be presented as one or more texts displayed or played on the user device, one or more graphical illustrations displayed on the user device, a content card comprising one or more texts displayed on the user device, a content card comprising integrated texts and graphical illustrations displayed on the user device, or any combinations thereof. The statements may be generated with respect to different objectives, for example, education, feedback on performance, insight, motivation, etc. A statement provides valuable feedback and inspiration to the user, and helps the user to choose new opportunities to form healthier behavior and habits. Accordingly, the micromodule management system can provide to the user, insightful information that is personalized for each individual user and has more impact on the behavior of the user given its reliance of various statistics and historical and user data.

Note that use of the terms, node or nodes, refers to logical constructs for describing the data structures (of the statements) and facilitating an understanding of the chaining of the data structures that comprise the statements into a micromodule.

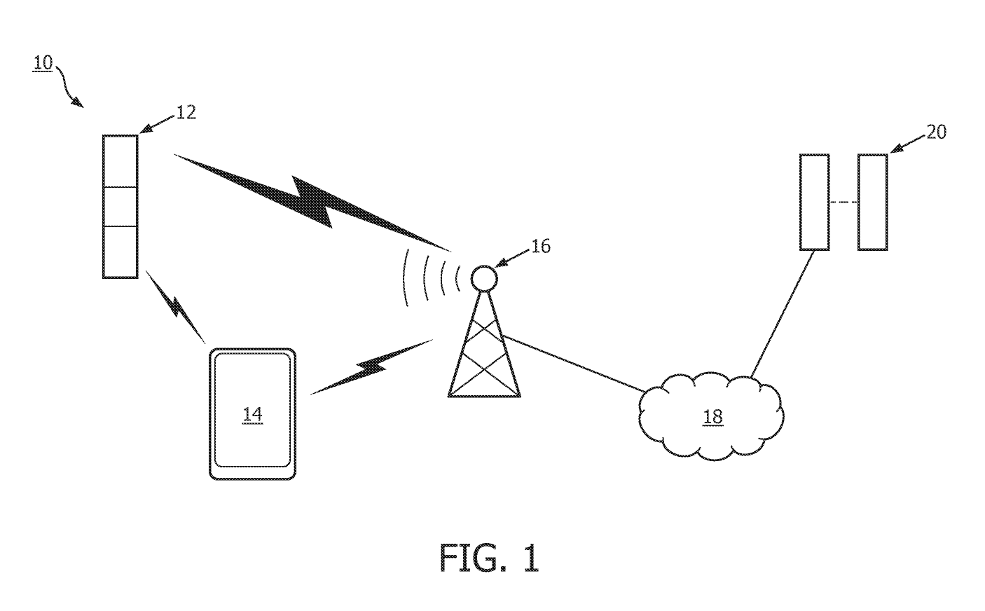

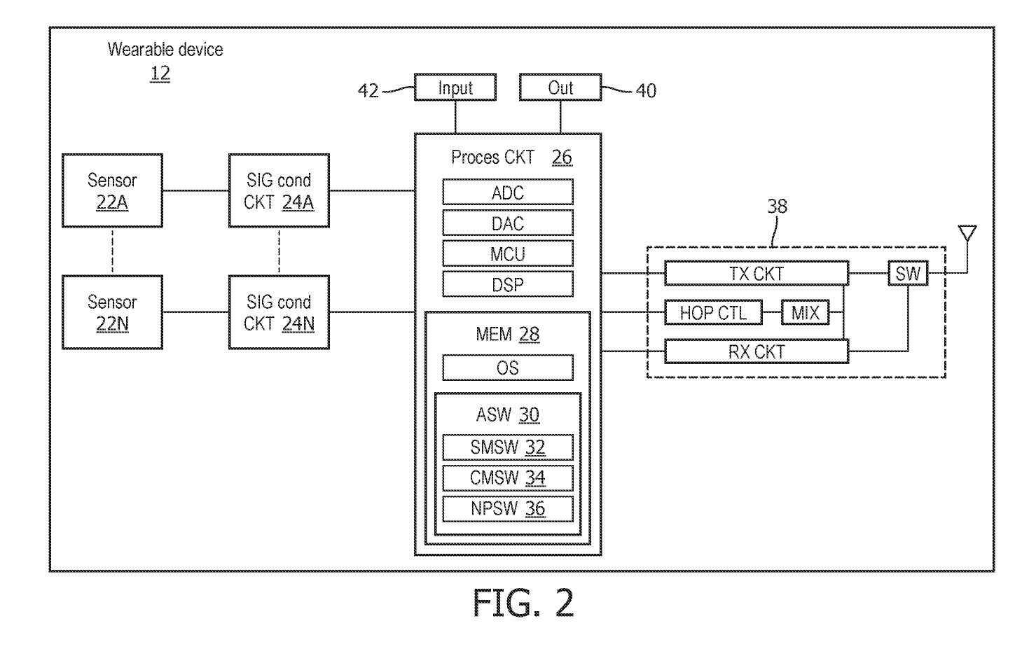

Referring now to FIG. 1, shown is an example environment 10 in which certain embodiments of a micromodule management system may be implemented. It should be appreciated by one having ordinary skill in the art in the context of the present disclosure that the environment 10 is one example among many, and that some embodiments of a micromodule management system may be used in environments with fewer, greater, and/or different components that those depicted in FIG. 1. The environment 10 comprises a plurality of devices that enable communication of information throughout one or more networks. The depicted environment 10 comprises a wearable device 12, an electronics (portable) device 14, a cellular/wireless network 16, a wide area network 18 (e.g., also described herein as the Internet), and a remote computing system 20. Note that the wearable device 12 and the electronics device 14 are also referred to as user devices. The wearable device 12, as described further in association with FIG. 2, is typically worn by the user (e.g., around the wrist or torso or attached to an article of clothing, or may include Google® glasses, wearable lenses, etc. using real time image capture, virtual, or augmented reality), and comprises a plurality of sensors that track physical activity of the user (e.g., steps, swim strokes, pedaling strokes, etc.), sense/measure or derive physiological parameters (e.g., heart rate, respiration, skin temperature, etc.) based on the sensor data, and optionally sense various other parameters (e.g., outdoor temperature, humidity, location, etc.) pertaining to the surrounding environment of the wearable device 12. For instance, in some embodiments, the wearable device 12 may comprise a global navigation satellite system (GNSS) receiver, including a GPS receiver, which tracks and provides location coordinates for the device 12. In some embodiments, the wearable device 12 may comprise indoor location technology, including beacons, RFID or other coded light technologies, WiFi, etc. Some embodiments of the wearable device 12 may include a motion or inertial tracking sensor, including an accelerometer and/or a gyroscope, providing movement data of the user. A representation of such gathered data may be communicated to the user via an integrated display on the wearable device 12 and/or on another device or devices.

Also, such data gathered by the wearable device 12 may be communicated (e.g., continually, periodically, and/or aperiodically, including upon request) to one or more electronics devices, such as the electronics device 14 or via the cellular/wireless network 16 to the computing system 20. Such communication may be achieved wirelessly (e.g., using near field communications (NFC) functionality, Bluetooth functionality, 802.11-based technology, etc.) and/or according to a wired medium (e.g., universal serial bus (USB), etc.). Further discussion of the wearable device 12 is described below in association with FIG. 2.

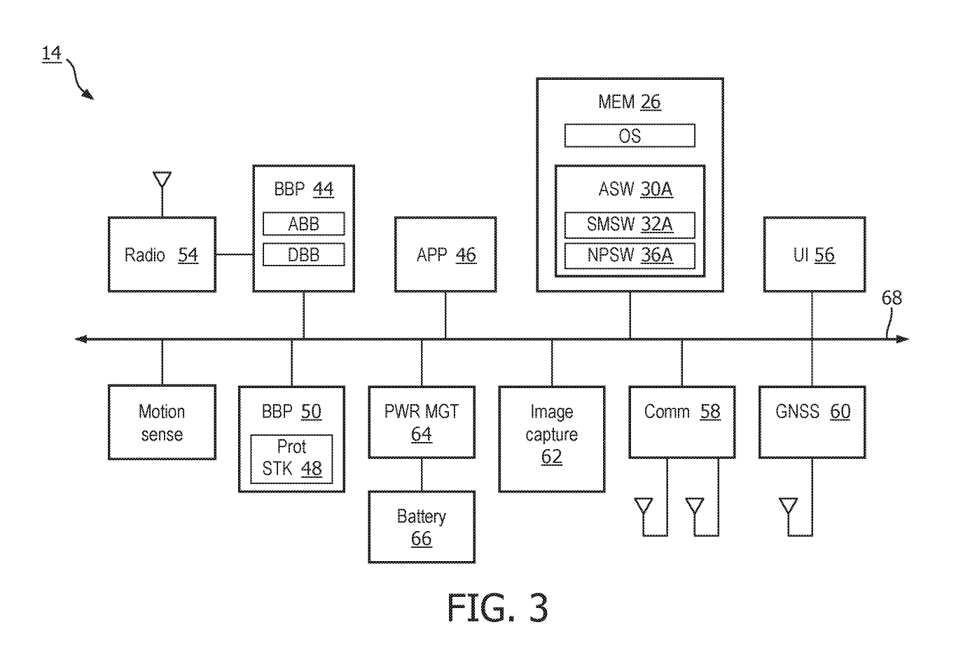

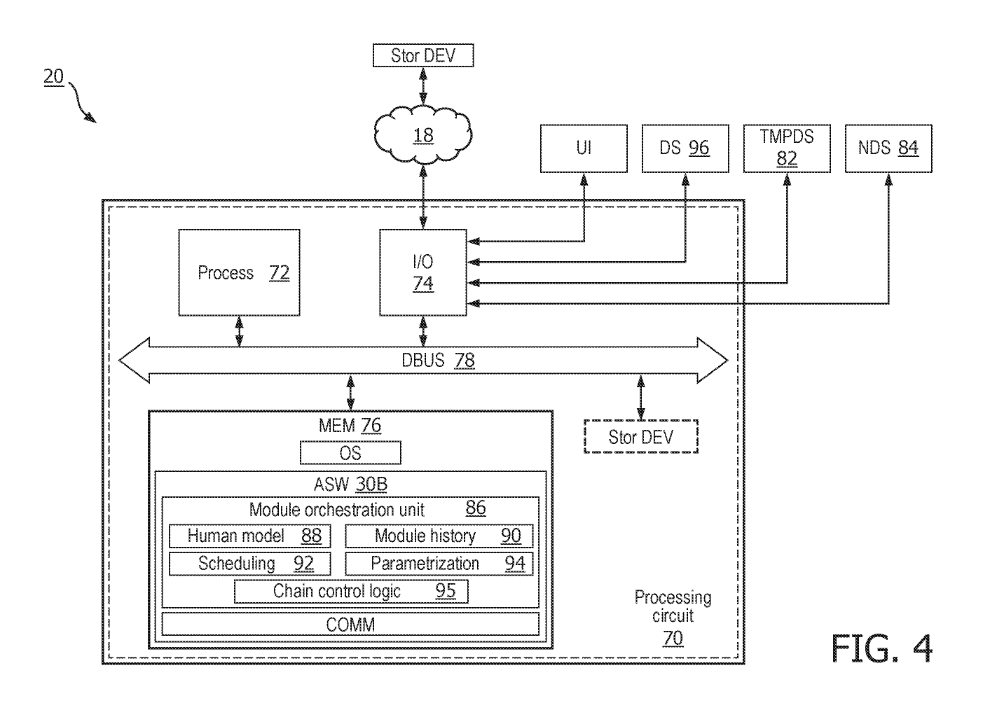

The electronics device 14 may be embodied as a smartphone, mobile phone, cellular phone, pager, stand-alone image capture device (e.g., camera), laptop, workstation, among other handheld and portable computing/communication devices, including communication devices having wireless communication capability, including telephony functionality. It is noted that if the electronics device 14 is embodied as a laptop or computer in general, the architecture more resembles that of the computing system 20 shown and described in association with FIG. 4. In some embodiments, the electronics device 14 may have built-in, image capture/recording functionality. In the depicted embodiment of FIG. 1, the electronics device 14 is a smartphone, though it should be appreciated that the electronics device 14 may take the form of other types of devices as described above. Further discussion of the electronics device 14 is described below in association with FIG. 3.

The cellular/wireless network 16 may include the necessary infrastructure to enable cellular and/or wireless communications by the electronics device 14 and optionally the wearable device 12. There are a number of different digital cellular technologies suitable for use in the cellular network 16, including: GSM, GPRS, CDMAOne, CDMA2000, Evolution-Data Optimized (EV-DO), EDGE, Universal Mobile Telecommunications System (UMTS), Digital Enhanced Cordless Telecommunications (DECT), Digital AMPS (IS-136/TDMA), and Integrated Digital Enhanced Network (iDEN), among others. The cellular/wireless network 16 may include modems, routers, etc. to enable the wearable device 12 and/or electronics device 14 to access the Internet via a wireless network, including according to wireless fidelity (WiFi) specifications.

The wide area network 18 may comprise one or a plurality of networks that in whole or in part comprise the Internet. The electronics device 14 and optionally wearable device 12 access one or more of the devices of the computing system 20 via the Internet 18, which may be further enabled through access to one or more networks including PSTN (Public Switched Telephone Networks), POTS, Integrated Services Digital Network (ISDN), Ethernet, Fiber, DSL/ADSL, WiFi, among others.

The computing system 20 comprises one or more devices coupled to the wide area network 18, including one or more computing devices networked together, including an application server(s) and data storage. The computing system 20 may serve as a cloud computing environment (or other server network) for the electronics device 14 and/or wearable device 12, performing processing and data storage on behalf of (or in some embodiments, in addition to) the electronics devices 14 and/or wearable device 12. In one embodiment, the computing system 20 may be configured to be a backend server for a health program. The computing system 20 receives data collected via one or more of the wearable device 12 or electronics device 14 and/or other devices or applications, stores the received data in a data structure (e.g., user profile database, etc.), and generates the notifications for presentation to the user. The computing system 20 is programmed to handle the operations of one or more health or wellness programs implemented on the wearable device 12 and/or electronics device 14 via the networks 16 and/or 18. For example, the computing system 20 processes user registration requests, user device activation requests, user information updating requests, data uploading requests, data synchronization requests, etc. The data received at the computing system 20 may be a plurality of measurements pertaining to the parameters, for example, body movements and activities, heart rate, respiration rate, blood pressure, body temperature, light and visual information, etc. and the corresponding context. In some embodiments, the data received at the computing system 20 may include measured, monitored, and/or inputted interactions between two or more individuals, including mother and a baby. Such data may further be received from social media websites. Based on the data observed during a period of time and/or over a large population of users, the computing system 20 generates statements pertaining to each specific parameter, and provides the statements via the networks 16 and/or 18 as an ongoing narrative of statements or notifications for presentation on devices 12 and/or 14. In some embodiments, the computing system 20 is configured to be a backend server for a health-related program or a health-related application implemented on the mobile devices. The functions of the computing system 20 described above are for illustrative purpose only. The present disclosure is not intended to be limiting. The computing system 20 may be a general computing server or a dedicated computing server. The computing system 20 may be configured to provide backend support for a program developed by a specific manufacturer.

When embodied as a cloud service or services, the computing system 20 may comprise an internal cloud, an external cloud, a private cloud, or a public cloud (e.g., commercial cloud). For instance, a private cloud may be implemented using a variety of cloud systems including, for example, Eucalyptus Systems, VMWare vSphere®, or Microsoft® HyperV. A public cloud may include, for example, Amazon EC2®, Amazon Web Services®, Terremark®, Savvis®, or GoGrid®. Cloud-computing resources provided by these clouds may include, for example, storage resources (e.g., Storage Area Network (SAN), Network File System (NFS), and Amazon S3®), network resources (e.g., firewall, load-balancer, and proxy server), internal private resources, external private resources, secure public resources, infrastructure-as-a-services (IaaSs), platform-as-a-services (PaaSs), or software-as-a-services (SaaSs). The cloud architecture of the computing system 20 may be embodied according to one of a plurality of different configurations. For instance, if configured according to MICROSOFT AZURE™, roles are provided, which are discrete scalable components built with managed code. Worker roles are for generalized development, and may perform background processing for a web role. Web roles provide a web server and listen and respond for web requests via an HTTP (hypertext transfer protocol) or HTTPS (HTTP secure) endpoint. VM roles are instantiated according to tenant defined configurations (e.g., resources, guest operating system). Operating system and VM updates are managed by the cloud. A web role and a worker role run in a VM role, which is a virtual machine under the control of the tenant. Storage and SQL services are available to be used by the roles. As with other clouds, the hardware and software environment or platform, including scaling, load balancing, etc., are handled by the cloud.

In some embodiments, the computing system 20 may be configured into multiple, logically-grouped servers, referred to as a server farm. The computing system 20 may comprise plural server devices geographically dispersed, administered as a single entity, or distributed among a plurality of server farms, executing one or more applications on behalf of one or more of the devices 12 and/or 14. The devices of the computing system 20 within each farm may be heterogeneous. One or more of the devices of the computing system 20 may operate according to one type of operating system platform (e.g., WINDOWS NT, manufactured by Microsoft Corp. of Redmond, Wash.), while one or more of the other devices of the computing system 20 may operate according to another type of operating system platform (e.g., Unix or Linux). The devices of the computing system 20 may be logically grouped as a server farm that may be interconnected using a wide-area network (WAN) connection or medium-area network (MAN) connection. The devices of the computing system 20 may each be referred to as, and operate according to, a file server device, application server device, web server device, proxy server device, or gateway server device. In one embodiment, the computing system 20 provide an API or web interface that enables the devices 12 and/or 14 to communicate with the computing system 20. The computing system 20 may also be configured to be interoperable across other servers and generate statements in a format that is compatible with other programs. In some embodiments, one or more of the functionality of the computing system 20 may be performed at the respective devices 12 and/or 14. Further discussion of the computing system 20 is described below in association with FIG. 4.

An embodiment of a micromodule management system may comprise the wearable device 12, the electronics device 14, and/or the computing system 20. In other words, one or more of the aforementioned devices 12, 14, and 20 may implement the functionality of the micromodule management system. For instance, the wearable device 12 may comprise all of the functionality of a micromodule management system, enabling the user to avoid the need for Internet connectivity and/or carrying a smartphone 14 around. In some embodiments, the functionality of the micromodule management system may be implemented using a combination of the wearable device 12 and the electronics device 14 and/or the computing system 20 (with or without the electronics device 14). For instance, the wearable device 12 and/or the electronics device 14 may present notifications via a user interface and provide sensing functionality, yet rely on remote data structures of the computing system 20 and remote processing of the computing systems 20.

As an example, the wearable device 12 may monitor activity of the user, and communicate context and the sensed parameters (e.g., location coordinates, motion data, physiological data, etc.) to one of the devices (e.g., the electronics device 14 and/or the computing system 20) external to the wearable device 12, the computing system 20 where all statement generation and publish selection is performed, and then each notification may be generated at one of the devices remote to the wearable device 12 and communicated back to the wearable device 12 for presentation according to a given temporal order (e.g., at different time intervals) relative to the presentation of other notifications. One benefit to the latter embodiment is that off-loading of the computational resources of the wearable device 12 is enabled, conserving power consumed by the wearable device 12. In some embodiments, the notifications may be presented by the wearable device 12 and/or the electronics device 14 and all other processing may be performed by the computing system 20, and in some embodiments, the notifications may be presented by the wearable device 12 and/or the electronics device 14 and all other processing performed by the electronics device 14, and in some embodiments, the notifications and processing may be entirely performed by the wearable device 12 and/or the electronics device 14. These and/or other variations are contemplated to be within the scope of the disclosure. For instance, in some embodiments, networks and devices associated with the micromodule management system may be configured to be the same for all users, or customized for a sub-population, including created separately for each user.

Attention is now directed to FIG. 2, which illustrates an example wearable device 12 in which all or a portion of the functionality of a micromodule management system may be implemented. That is, FIG. 2 illustrates an example architecture (e.g., hardware and software) for the example wearable device 12. It should be appreciated by one having ordinary skill in the art in the context of the present disclosure that the architecture of the wearable device 12 depicted in FIG. 2 is but one example, and that in some embodiments, additional, fewer, and/or different components may be used to achieve similar and/or additional functionality. In one embodiment, the wearable device 12 comprises a plurality of sensors 22 (e.g., 22A-22N), one or more signal conditioning circuits 24 (e.g., SIG COND CKT 24A-SIG COND CKT 24N) coupled respectively to the sensors 22, and a processing circuit 26 (PROCES CKT) that receives the conditioned signals from the signal conditioning circuits 24. In one embodiment, the processing circuit 26 comprises an analog-to-digital converter (ADC), a digital-to-analog converter (DAC), a microcontroller unit (MCU), a digital signal processor (DSP), and memory (MEM) 28. In some embodiments, the processing circuit 26 may comprise fewer or additional components than those depicted in FIG. 2. For instance, in one embodiment, the processing circuit 26 may consist of the microcontroller. In some embodiments, the processing circuit 26 may include the signal conditioning circuits 24. The memory 28 comprises an operating system (OS) and application software (ASW) 30.

The application software 30 comprises a plurality of software modules (e.g., executable code/instructions) including sensor measurement software (SMSW) 32, communications software (CMSW) 34, and notification presentation software (NPSW) 36. In some embodiments, the application software 30 may include additional software that implements some or all of the processing functionality of a micromodule management system, including the tracking of statistics for the micromodules, human behavior modeling, scheduling of the micromodules, and parametrization. For purposes of brevity, the description about the application software 30 hereinafter is premised on the assumption that the various processing performed by the micromodule management system is implemented at the computing device 20, and that the presentation of the notifications based on the processing is performed at the wearable device 12 (and/or electronics device 14, FIG. 1) during non-overlapping intervals based on the current context from the computing system 20 directly or via communications with the electronics device 14 (which receives the notifications from the computing system 20). The sensor measurement software 32 comprises executable code to process the signals (and associated data) measured by the sensors 22 and record and/or derive physiological parameters, such as heart rate, blood pressure, respiration, perspiration, etc. and movement and/or location data.

The communications software 34 comprises executable code/instructions to enable a communications circuit 38 of the wearable device 12 to operate according to one or more of a plurality of different communication technologies (e.g., NFC, Bluetooth, WiFi, including 802.11, GSM, LTE, CDMA, WCDMA, Zigbee, etc.). The communications software 34 instructs and/or controls the communications circuit 38 to transmit the raw sensor data and/or the derived information from the sensor data to the computing system 20 (e.g., directly via the cellular/wireless network 16, or indirectly via the electronics device 14). The communications software 34 may also include browser software in some embodiments to enable Internet connectivity. The communications software 34 may also be used to access certain services, such as mapping/place location services, which may be used to determine context for the sensor data. These services may be used in some embodiments of a micromodule management system, and in some instances, may not be used. In some embodiments, the communications software 34 may be external to the application software 30 or in other segments of memory. The notification presentation software 36 is configured to receive the notifications via the communications software 34 and communications circuit 38 as the notifications are communicated at different (non-overlapping) intervals based on the context (e.g., determined by the computing system 20 from the input data received from the wearable device 12). The notification presentation software 36 may format and present the notifications at an output interface 40 of the wearable device 12 at a time corresponding to when the notifications are received from the computing system 20 and/or electronics device 14 and/or at other times during the day or evening if different than when received. In some embodiments, the notification presentation software 36 may learn (e.g., based on previous notifications that were indicated, such as via feedback or use or neglect of similar and/or previous notifications) a preferred or best moment to present a current notification received from the computing system 20.

As indicated above, in one embodiment, the processing circuit 26 is coupled to the communications circuit 38. The communications circuit 38 serves to enable wireless communications between the wearable device 12 and other devices, including the electronics device 14 and the computing system 20, among other devices. The communications circuit 38 is depicted as a Bluetooth circuit, though not limited to this transceiver configuration. For instance, in some embodiments, the communications circuit 38 may be embodied as any one or a combination of an NFC circuit, WiFi circuit, transceiver circuitry based on Zigbee, 802.11, GSM, LTE, CDMA, WCDMA, among others such as optical or ultrasonic based technologies. The processing circuit 26 is further coupled to input/output (I/O) devices or peripherals, including an input interface 42 (INPUT) and the output interface 40 (OUT). Note that in some embodiments, functionality for one or more of the aforementioned circuits and/or software may be combined into fewer components/modules, or in some embodiments, further distributed among additional components/modules or devices. For instance, the processing circuit 26 may be packaged as an integrated circuit that includes the microcontroller (microcontroller unit or MCU), the DSP, and memory 28, whereas the ADC and DAC may be packaged as a separate integrated circuit coupled to the processing circuit 26. In some embodiments, one or more of the functionality for the above-listed components may be combined, such as functionality of the DSP performed by the microcontroller.

The sensors 22 are selected to perform detection and measurement of a plurality of physiological and behavioral parameters (e.g., typical behavioral parameters or activities including walking, running, cycling, and/or other activities, including shopping, walking a dog, working in the garden, etc.), including heart rate, heart rate variability, heart rate recovery, blood flow rate, activity level, muscle activity (e.g., movement of limbs, repetitive movement, core movement, body orientation/position, power, speed, acceleration, etc.), muscle tension, blood volume, blood pressure, blood oxygen saturation, respiratory rate, perspiration, skin temperature, body weight, and body composition (e.g., body mass index or BMI). At least one of the sensors 22 may be embodied as movement detecting sensors, including inertial sensors (e.g., gyroscopes, single or multi-axis accelerometers, such as those using piezoelectric, piezoresistive or capacitive technology in a microelectromechanical system (MEMS) infrastructure for sensing movement) and/or as GNSS sensors, including a GPS receiver to facilitate determinations of distance, speed, acceleration, location, altitude, etc. (e.g., location data, or generally, sensing movement), in addition to or in lieu of the accelerometer/gyroscope and/or indoor tracking (e.g., ibeacons, WiFi, coded-light based technology, etc.). The sensors 22 may also include flex and/or force sensors (e.g., using variable resistance), electromyographic sensors, electrocardiographic sensors (e.g., EKG, ECG) magnetic sensors, photoplethysmographic (PPG) sensors, bio-impedance sensors, infrared proximity sensors, acoustic/ultrasonic/audio sensors, a strain gauge, galvanic skin/sweat sensors, pH sensors, temperature sensors, pressure sensors, and photocells. The sensors 22 may include other and/or additional types of sensors for the detection of, for instance, barometric pressure, humidity, outdoor temperature, etc. In some embodiments, GNSS functionality may be achieved via the communications circuit 38 or other circuits coupled to the processing circuit 26.

The signal conditioning circuits 24 include amplifiers and filters, among other signal conditioning components, to condition the sensed signals including data corresponding to the sensed physiological parameters and/or location signals before further processing is implemented at the processing circuit 26. Though depicted in FIG. 2 as respectively associated with each sensor 22, in some embodiments, fewer signal conditioning circuits 24 may be used (e.g., shared for more than one sensor 22). In some embodiments, the signal conditioning circuits 24 (or functionality thereof) may be incorporated elsewhere, such as in the circuitry of the respective sensors 22 or in the processing circuit 26 (or in components residing therein). Further, although described above as involving unidirectional signal flow (e.g., from the sensor 22 to the signal conditioning circuit 24), in some embodiments, signal flow may be bi-directional. For instance, in the case of optical measurements, the microcontroller may cause an optical signal to be emitted from a light source (e.g., light emitting diode(s) or LED(s)) in or coupled to the circuitry of the sensor 22, with the sensor 22 (e.g., photocell) receiving the reflected/refracted signals.

The communications circuit 38 is managed and controlled by the processing circuit 26 (e.g., executing the communications software 34). The communications circuit 38 is used to wirelessly interface with the electronics device 14 (FIG. 3) and/or one or more devices of the computing system 20. In one embodiment, the communications circuit 38 may be configured as a Bluetooth (including BTLE) transceiver, though in some embodiments, other and/or additional technologies may be used, such as Wi-Fi, GSM, LTE, CDMA and its derivatives, Zigbee, NFC, among others. In the embodiment depicted in FIG. 2, the communications circuit 38 comprises a transmitter circuit (TX CKT), a switch (SW), an antenna, a receiver circuit (RX CKT), a mixing circuit (MIX), and a frequency hopping controller (HOP CTL). The transmitter circuit and the receiver circuit comprise components suitable for providing respective transmission and reception of an RF signal, including a modulator/demodulator, filters, and amplifiers. In some embodiments, demodulation/modulation and/or filtering may be performed in part or in whole by the DSP. The switch switches between receiving and transmitting modes. The mixing circuit may be embodied as a frequency synthesizer and frequency mixers, as controlled by the processing circuit 26. The frequency hopping controller controls the hopping frequency of a transmitted signal based on feedback from a modulator of the transmitter circuit. In some embodiments, functionality for the frequency hopping controller may be implemented by the microcontroller or DSP. Control for the communications circuit 38 may be implemented by the microcontroller, the DSP, or a combination of both. In some embodiments, the communications circuit 38 may have its own dedicated controller that is supervised and/or managed by the microcontroller.

In one example operation, a signal (e.g., at 2.4 GHz) may be received at the antenna and directed by the switch to the receiver circuit. The receiver circuit, in cooperation with the mixing circuit, converts the received signal into an intermediate frequency (IF) signal under frequency hopping control attributed by the frequency hopping controller and then to baseband for further processing by the ADC. On the transmitting side, the baseband signal (e.g., from the DAC of the processing circuit 26) is converted to an IF signal and then RF by the transmitter circuit operating in cooperation with the mixing circuit, with the RF signal passed through the switch and emitted from the antenna under frequency hopping control provided by the frequency hopping controller. The modulator and demodulator of the transmitter and receiver circuits may be frequency shift keying (FSK) type modulation/demodulation, though not limited to this type of modulation/demodulation, which enables the conversion between IF and baseband. In some embodiments, demodulation/modulation and/or filtering may be performed in part or in whole by the DSP. The memory 28 stores the communications software 34, which when executed by the microcontroller, controls the Bluetooth (and/or other protocols) transmission/reception.

Though the communications circuit 38 is depicted as an IF-type transceiver, in some embodiments, a direct conversion architecture may be implemented. As noted above, the communications circuit 38 may be embodied according to other and/or additional transceiver technologies.

The processing circuit 26 is depicted in FIG. 2 as including the ADC and DAC. For sensing functionality, the ADC converts the conditioned signal from the signal conditioning circuit 24 and digitizes the signal for further processing by the microcontroller and/or DSP. The ADC may also be used to convert analogs inputs that are received via the input interface 42 to a digital format for further processing by the microcontroller. The ADC may also be used in baseband processing of signals received via the communications circuit 38. The DAC converts digital information to analog information. Its role for sensing functionality may be to control the emission of signals, such as optical signals or acoustic signal, from the sensors 22. The DAC may further be used to cause the output of analog signals from the output interface 40. Also, the DAC may be used to convert the digital information and/or instructions from the microcontroller and/or DSP to analog signal that are fed to the transmitter circuit. In some embodiments, additional conversion circuits may be used.

The microcontroller and the DSP provide the processing functionality for the wearable device 12. In some embodiments, functionality of both processors may be combined into a single processor, or further distributed among additional processors. The DSP provides for specialized digital signal processing, and enables an offloading of processing load from the microcontroller. The DSP may be embodied in specialized integrated circuit(s) or as field programmable gate arrays (FPGAs). In one embodiment, the DSP comprises a pipelined architecture, with comprises a central processing unit (CPU), plural circular buffers and separate program and data memories according to a Harvard architecture. The DSP further comprises dual busses, enabling concurrent instruction and data fetches. The DSP may also comprise an instruction cache and I/O controller, such as those found in Analog Devices SHARC® DSPs, though other manufacturers of DSPs may be used (e.g., Freescale multi-core MSC81xx family, Texas Instruments C6000 series, etc.). The DSP is generally utilized for math manipulations using registers and math components that may include a multiplier, arithmetic logic unit (ALU, which performs addition, subtraction, absolute value, logical operations, conversion between fixed and floating point units, etc.), and a barrel shifter. The ability of the DSP to implement fast multiply-accumulates (MACs) enables efficient execution of Fast Fourier Transforms (FFTs) and Finite Impulse Response (FIR) filtering. Some or all of the DSP functions may be performed by the microcontroller. The DSP generally serves an encoding and decoding function in the wearable device 12. For instance, encoding functionality may involve encoding commands or data corresponding to transfer of information to the electronics device 14 or a device of the computing system 20. Also, decoding functionality may involve decoding the information received from the sensors 22 (e.g., after processing by the ADC).

The microcontroller comprises a hardware device for executing software/firmware, particularly that stored in memory 28. The microcontroller can be any custom made or commercially available processor, a central processing unit (CPU), a semiconductor based microprocessor (in the form of a microchip or chip set), a macroprocessor, or generally any device for executing software instructions. Examples of suitable commercially available microprocessors include Intel's® Itanium® and Atom® microprocessors, to name a few non-limiting examples. The microcontroller provides for management and control of the wearable device 12, including determining physiological parameters or location coordinates based on the sensors 22, and for enabling communication with the electronics device 14 and/or a device of the computing system 20, and for the presentation of a chain of notifications for the micromodule management system.

The memory 28 can include any one or combination of volatile memory elements (e.g., random access memory (RAM, such as DRAM, SRAM, SDRAM, etc.)) and nonvolatile memory elements (e.g., ROM, Flash, solid state, EPROM, EEPROM, etc.). Moreover, the memory 28 may incorporate electronic, magnetic, and/or other types of storage media.

The software in memory 28 may include one or more separate programs, each of which comprises an ordered listing of executable instructions for implementing logical functions. In the example of FIG. 2, the software in the memory 28 includes a suitable operating system and the application software 30, which includes a plurality of software modules 32-36 for implementing certain embodiments of a micromodule management system and algorithms for determining physiological and/or behavioral measures and/or other information (e.g., including location, speed of travel, etc.) based on the output from the sensors 22. The raw data from the sensors 22 may be used by the algorithms to determine various physiological and/or behavioral measures (e.g., heart rate, biomechanics, such as swinging of the arms), and may also be used to derive other parameters, such as energy expenditure, heart rate recovery, aerobic capacity (e.g., VO2 max, etc.), among other derived measures of physical performance. In some embodiments, these derived parameters may be computed externally (e.g., at the electronics devices 14 or one or more devices of the computing system 20) in lieu of, or in addition to, the computations performed local to the wearable device 12. In some embodiments, the GPS functionality of the sensors 22 collects contextual data (time and location data, including location coordinates). The application software 30 may collect location data by sampling the location readings from the sensor 22 over a period of time (e.g., minutes, hours, days, weeks, etc.). The application software 30 may also collect information about the means of ambulation. For instance, the GPS data (which may include time coordinates) may be used by the application software 30 to determine speed of travel, which may indicate whether the user is moving within a vehicle, on a bicycle, or walking or running. In some embodiments, other and/or additional data may be used to assess the type of activity, including physiological data (e.g., heart rate, respiration rate, galvanic skin response, etc.) and/or behavioral data.

The operating system essentially controls the execution of other computer programs, such as the application software 30 and associated modules 32-36, and provides scheduling, input-output control, file and data management, memory management, and communication control and related services. The memory 28 may also include user data, including weight, height, age, gender, goals, body mass index (BMI) that are used by the microcontroller executing the executable code of the algorithms to accurately interpret the measured physiological and/or behavioral data. The user data may also include historical data relating past recorded data to prior contexts.

Although the application software 30 (and component parts 32-36) are described above as implemented in the wearable device 12, some embodiments may distribute the corresponding functionality among the wearable device 12 and other devices (e.g., electronics device 14 and/or one or more devices of the computing system 20), or in some embodiments, the application software 30 (and component parts 32-36) may be implemented in another device (e.g., the electronics device 14).

The software in memory 28 comprises a source program, executable program (object code), script, or any other entity comprising a set of instructions to be performed. When a source program, then the program may be translated via a compiler, assembler, interpreter, or the like, so as to operate properly in connection with the operating system. Furthermore, the software can be written as (a) an object oriented programming language, which has classes of data and methods, or (b) a procedure programming language, which has routines, subroutines, and/or functions, for example but not limited to, C, C++, Python, Java, among others. The software may be embodied in a computer program product, which may be a non-transitory computer readable medium or other medium.

The input interface 42 comprises an interface (e.g., including a user interface) for entry of user input, such as a button or microphone or sensor (e.g., to detect user input) or touch-type display. In some embodiments, the input interface 42 may serve as a communications port for downloaded information to the wearable device 12 (such as via a wired connection). The output interfaces 40 comprises an interface for the presentation or transfer of data, including a user interface (e.g., display screen presenting a graphical user interface) or communications interface for the transfer (e.g., wired) of information stored in the memory, or to enable one or more feedback devices, such as lighting devices (e.g., LEDs), audio devices (e.g., tone generator and speaker), and/or tactile feedback devices (e.g., vibratory motor). For instance, the output interface 40 may be used to present the notifications to the user. In some embodiments, at least some of the functionality of the input and output interfaces 42 and 40, respectively, may be combined, including being embodied at least in part as a touch-type display screen for the entry of input (e.g., to select an opportunity for behavioral change, such as via a presented invite in a dashboard or other screen, to input preferences, etc.) and presentation of notifications, among other data. In some embodiments, selection may be made automatically after the invitation based on detecting the context of the user (e.g., a context aware feature).

Referring now to FIG. 3, shown is an example electronics device 14 in which all or a portion of the functionality of a micromodule management system may be implemented. Similar to the description for the wearable device 12 of FIG. 2, and for the sake of brevity, the application software of the electronics device 14 comprises similar components as that for the wearable device 12, with the understanding that fewer or a greater number of software modules of the micromodule management system may be used in some embodiments. In the depicted example, the electronics device 14 is embodied as a smartphone (hereinafter, referred to smartphone 14), though in some embodiments, other types of devices may be used, such as a workstation, laptop, notebook, tablet, etc. It should be appreciated by one having ordinary skill in the art that the logical block diagram depicted in FIG. 3 and described below is one example, and that other designs may be used in some embodiments. The application software (ASW) 30A comprises a plurality of software modules (e.g., executable code/instructions) including sensor measurement software (SMSW) 32A and notification presentation software (NPSW) 36A, as well as communications software as is expected of mobile phones. In some embodiments, the application software 30A may include additional software that implements data structure definitions, associations, and additional processing. Note that the application software 30A (and component parts 32A and 36A) comprise at least some of the functionality of the application software 30 (and component parts 32 and 36) described above for the wearable device 12, and may include additional software pertinent to smartphone operations (e.g., possibly not found in wearable devices 12).

The smartphone 14 comprises at least two different processors, including a baseband processor (BBP) 44 and an application processor (APP) 46. As is known, the baseband processor 44 primarily handles baseband communication-related tasks and the application processor 46 generally handles inputs and outputs and all applications other than those directly related to baseband processing. The baseband processor 44 comprises a dedicated processor for deploying functionality associated with a protocol stack (PROT STK) 48, such as a GSM (Global System for Mobile communications) protocol stack, among other functions. The application processor 46 comprises a multi-core processor for running applications, including all or a portion of the application software 30A and its corresponding component parts 32A and 36A as described above in association with the wearable device 12 of FIG. 2. The baseband processor 44 and application processor 46 have respective associated memory (e.g., MEM) 50, 52, including random access memory (RAM), Flash memory, etc., and peripherals, and a running clock.

More particularly, the baseband processor 44 may deploy functionality of the protocol stack 48 to enable the smartphone 14 to access one or a plurality of wireless network technologies, including WCDMA (Wideband Code Division Multiple Access), CDMA (Code Division Multiple Access), EDGE (Enhanced Data Rates for GSM Evolution), GPRS (General Packet Radio Service), Zigbee (e.g., based on IEEE 802.15.4), Bluetooth, WiFi (Wireless Fidelity, such as based on IEEE 802.11), and/or LTE (Long Term Evolution), among variations thereof and/or other telecommunication protocols, standards, and/or specifications. The baseband processor 44 manages radio communications and control functions, including signal modulation, radio frequency shifting, and encoding. The baseband processor 44 comprises, or may be coupled to, a radio (e.g., RF front end) 54 and/or a GSM modem having one or more antennas, and analog and digital baseband circuitry (ABB, DBB, respectively in FIG. 3). The radio 54 comprises a transceiver and a power amplifier to enable the receiving and transmitting of signals of a plurality of different frequencies, enabling access to the cellular network 16 (FIG. 1), and hence the communication of user data and associated contexts to the computing system 20 (FIG. 1) and the receipt of notifications from the computing system 20.

The analog baseband circuitry is coupled to the radio 54 and provides an interface between the analog and digital domains of the GSM modem. The analog baseband circuitry comprises circuitry including an analog-to-digital converter (ADC) and digital-to-analog converter (DAC), as well as control and power management/distribution components and an audio codec to process analog and/or digital signals received indirectly via the application processor 46 or directly from the smartphone user interface 56 (e.g., microphone, earpiece, ring tone, vibrator circuits, etc.). The ADC digitizes any analog signals for processing by the digital baseband circuitry. The digital baseband circuitry deploys the functionality of one or more levels of the GSM protocol stack (e.g., Layer 1, Layer 2, etc.), and comprises a microcontroller (e.g., microcontroller unit or MCU, also referred to herein as a processor) and a digital signal processor (DSP, also referred to herein as a processor) that communicate over a shared memory interface (the memory comprising data and control information and parameters that instruct the actions to be taken on the data processed by the application processor 46). The MCU may be embodied as a RISC (reduced instruction set computer) machine that runs a real-time operating system (RTIOS), with cores having a plurality of peripherals (e.g., circuitry packaged as integrated circuits) such as RTC (real-time clock), SPI (serial peripheral interface), I2C (inter-integrated circuit), UARTs (Universal Asynchronous Receiver/Transmitter), devices based on IrDA (Infrared Data Association), SD/MMC (Secure Digital/Multimedia Cards) card controller, keypad scan controller, and USB devices, GPRS crypto module, TDMA (Time Division Multiple Access), smart card reader interface (e.g., for the one or more SIM (Subscriber Identity Module) cards), timers, and among others. For receive-side functionality, the MCU instructs the DSP to receive, for instance, in-phase/quadrature (I/Q) samples from the analog baseband circuitry and perform detection, demodulation, and decoding with reporting back to the MCU. For transmit-side functionality, the MCU presents transmittable data and auxiliary information to the DSP, which encodes the data and provides to the analog baseband circuitry (e.g., converted to analog signals by the DAC).

The application processor 46 operates under control of an operating system (OS) that enables the implementation of a plurality of user applications, including the application software 30A. The application processor 46 may be embodied as a System on a Chip (SOC), and supports a plurality of multimedia related features including web browsing to access one or more computing devices of the computing system 20 (FIG. 4) that are coupled to the Internet, email, multimedia entertainment, games, etc. For instance, the application processor 46 may execute interface software (e.g., middleware, such as a browser with or operable in association with one or more application program interfaces (APIs)) to enable access to a cloud computing framework or other networks to provide remote data access/storage/processing, and through cooperation with an embedded operating system, access to calendars, location services, reminders, etc. For instance, in some embodiments, the micromodule management system may operate using cloud computing, where the processing of sensor data (e.g., location data, including data received from the wearable device 12 or from integrated sensors within the smartphone 14, including motion sense, image capture, location detect, etc.) and context may be achieved by one or more devices of the computing system 20. The application processor 46 generally comprises a processor core (Advanced RISC Machine or ARM), and further comprises or may be coupled to multimedia modules (for decoding/encoding pictures, video, and/or audio), a graphics processing unit (GPU), communication interfaces (COMM) 58, and device interfaces. The communication interfaces 58 may include wireless interfaces, including a Bluetooth (BT) (and/or Zigbee in some embodiments) module that enables wireless communication with an electronics device, including the wearable device 12, other electronics devices, and a Wi-Fi module for interfacing with a local 802.11 network. The application processor 46 further comprises, or is coupled to, a global navigation satellite systems (GNSS) transceiver or receiver (GNSS) 60 for access to a satellite network to, for instance, provide location services.

The device interfaces coupled to the application processor 46 may include the user interface 56, including a display screen. The display screen, similar to a display screen of the wearable device user interface, may be embodied in one of several available technologies, including LCD or Liquid Crystal Display (or variants thereof, such as Thin Film Transistor (TFT) LCD, In Plane Switching (IPS) LCD)), light-emitting diode (LED)-based technology, such as organic LED (OLED), Active-Matrix OLED (AMOLED), or retina or haptic-based technology. For instance, the display screen may be used to present web pages, dashboards, notifications, and/or other documents or data received from the computing system 20 and/or the display screen may be used to present information (e.g., notifications) in graphical user interfaces (GUIs) rendered locally in association with the application software 30A. In some embodiments, information may be presented as part of a speech communication in a spoken dialogue system. Other user interfaces 56 include a keypad, microphone, speaker, ear piece connector, I/O interfaces (e.g., USB (Universal Serial Bus)), SD/MMC card, among other peripherals. Also coupled to the application processor 46 is an image capture device (IMAGE CAPTURE) 62. The image capture device 62 comprises an optical sensor (e.g., a charged coupled device (CCD) or a complementary metal-oxide semiconductor (CMOS) optical sensor). The image capture device 62 may be used to detect various physiological parameters of a user, including blood pressure based on remote photoplethysmography (PPG). Also included is a power management device 64 that controls and manages operations of a battery 66. The components described above and/or depicted in FIG. 3 share data over one or more busses, and in the depicted example, via data bus 68. It should be appreciated by one having ordinary skill in the art, in the context of the present disclosure, that variations to the above may be deployed in some embodiments to achieve similar functionality.

In the depicted embodiment, the application processor 46 runs the application software 30A, which in one embodiment, includes a plurality of software modules (e.g., executable code/instructions) including the sensor measurement software (SMSW) 32A and the notification presentation software (NPSW) 36A. Since the description of the application software 30 and software modules 32 and 36 has been described above in association with the wearable device 12 (FIG. 2), and since the same functionality is present in software 32A (albeit on perhaps different sensor data) and 36A, discussion of the same here is omitted for brevity. It is noteworthy, however, that some or all of the software functionality may be implemented in the smartphone 14. For instance, all of the functionality of the application software 30A may be implemented in the smartphone 14, or functionality of the application software 30A may be divided among plural devices of the environment 10 (FIG. 1) in some embodiments. The application software 30A may also comprises executable code to process the signals (and associated data) measured by the sensors (of the wearable device 12 as communicated to the smartphone 14, or based on sensors integrated within the smartphone 14) and record and/or derive physiological parameters, such as heart rate, blood pressure, respiration, perspiration, etc. Note that functionality of the software modules 32A and 36A, similar to those described for the wearable device 12, may be combined in some embodiments, or further distributed among additional modules. In some embodiments, the execution of the application software 30A and associated modules 32A and 36A may be distributed among plural devices, as set forth above. Note that all or a portion of the aforementioned hardware and/or software of the smartphone 14 may be referred to herein as a processing circuit.

Referring now to FIG. 4, shown is a computing system 20 in which at least a portion of the functionality of a micromodule management system may be implemented. The computing system 20 may comprise a single computing device as shown here, or in some embodiments, may comprise plural devices that at least collectively perform the functionality described below, among optionally other functions. In one embodiment, the computing system 20 may be embodied as an application server, a computer, among other computing devices. One having ordinary skill in the art should appreciate in the context of the present disclosure that the example computing system 20 is merely illustrative of one embodiment, and that some embodiments of computing devices may comprise fewer or additional components, and/or some of the functionality associated with the various components depicted in FIG. 4 may be combined, or further distributed among additional modules or computing devices, in some embodiments. The computing system 20 is depicted in this example as a computer system, including one providing a function of an application server. It should be appreciated that certain well-known components of computer systems are omitted here to avoid obfuscating relevant features of the computing system 20. In one embodiment, the computing system 20 comprises a processing circuit 70 comprising hardware and software components. In some embodiments, the processing circuit 70 may comprise additional components or fewer components. For instance, memory may be separate. The processing circuit 70 comprises one or more processors, such as processor 72 (PROCESS), input/output (I/O) interface(s) 74 (I/O), and memory 76 (MEM), all coupled to one or more data busses, such as data bus 78 (DBUS). The memory 76 may include any one or a combination of volatile memory elements (e.g., random-access memory RAM, such as DRAM, and SRAM, etc.) and nonvolatile memory elements (e.g., ROM, Flash, solid state, EPROM, EEPROM, hard drive, tape, CDROM, etc.).

The memory 76 may store a native operating system (OS), one or more native applications, emulation systems, or emulated applications for any of a variety of operating systems and/or emulated hardware platforms, emulated operating systems, etc. In some embodiments, the processing circuit 70 may include, or be coupled to, one or more separate storage devices. For instance, in the depicted embodiment, the processing circuit 70 is coupled via the I/O interfaces 74 to template data structures (TMPDS) 82 and notification data structures (NDS) 84. In some embodiments, the template data structures 82 and/or the notifications data structures 84 may be coupled to the processing circuit 70 via the data bus 78 or coupled to the processing circuit 70 via the I/O interfaces 74 as network-connected storage devices (STOR DEVS). The data structures 82 and/or 84 may be stored in persistent memory (e.g., optical, magnetic, and/or semiconductor memory and associated drives). In some embodiments, the data structures 82 and/or 84 may be stored in memory 76.

The template data structures 82 are configured to store one or more templates that are used in a statement definition stage to generate the statements conveying information to the user. The statements for different objectives may use different templates. For example, education related statements may apply templates with referral links to educational resources, feedback on performance may apply templates with rating/ranking comments, etc. The template data structures 82 may be maintained by an administrator operating the computing system 20. The template data structures 82 may be updated based on the usage of each template, the feedback on each generated statement, etc. The templates that are more often used and/or receive more positive feedbacks from the users may be highly recommended to generate the statements in the future. In some embodiments, the templates may be general templates that can be used to generate all types of statements. In some other embodiments, the templates may be classified into categories, each category pertaining to a parameter. For example, templates for generating statement pertaining to heart rate may be partially different from templates for generating statement pertaining to sleep quality. The notifications data structures 84 are configured to store the statements that are constructed based on the templates.

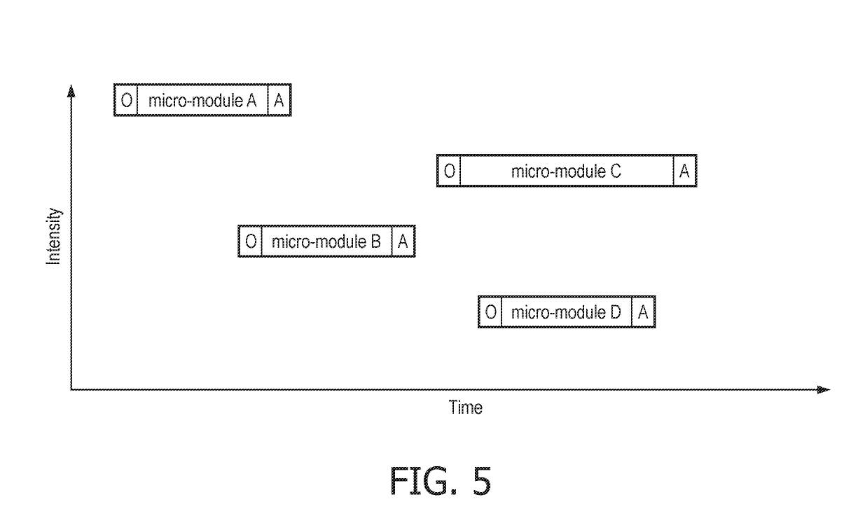

In the embodiment depicted in FIG. 4, the memory 76 comprises an operating system (OS) and the application software 30B. In some embodiments, an operating system may be omitted. The application software 30B comprises a module orchestration unit 86 and a communications module (COMM). Digressing briefly, and referring to FIG. 5, in contrast to the conventional brick-wall health program module structures described above, certain embodiments of a micromodule management system enables a more flexible arrangement of the management of micromodules, though not limited to the specific arrangement depicted in FIG. 5. In particular, FIG. 5 conceptually illustrates an open-ended service of micromodules that address the targets of a user, the service comprising a plurality of micromodules A-D (which may be of the same type—e.g., fitness program, or of different types—e.g., sleep training, fitness, etc.). The continuous service, which may be described as open-ended, contains information about the lifestyle, physiological, and psycho-social factors influencing the behavior of the user, and agreements on health targets of the user. The actual health interventions (electronic interventions, notifications, statements) are provided as micro-modules. Each micro-module starts with the detection of an opportunity (O) and ends with an assessment (A). That is, each micromodule concludes with an assessment, which is an end statement regarding the started chain. For instance, the assessment or statement typically regards performance in a specific chain.

Referring again to FIG. 4, with continued reference to FIG. 5, the module orchestration unit 86 selects which of one or more micromodules are to be offered to the user over time, and collects the results of the assessments. In the embodiment shown in FIG. 4, the module orchestration unit 86 comprises a human model component 88, a module history component 90, a scheduling component 92, a parametrization component 94, and chain control logic 95. The module orchestration unit 86 and components 88-95 comprise instructions (executable code) that, when executed by one or more processors (e.g., processor 72), implements the functionality of an embodiment of a micromodule management system. For instance, the selection process (selecting micromodules) is based on different software components, each of them responsible for at least a part of the scoring of each micromodule. Note that in some embodiments, functionality of two or more of the components 88-95 may be distributed among plural devices, or in some embodiments, condensed into fewer modules. The module history component 90 is where, in one embodiment, the assessments are stored. The module history component 90 keeps track of the statistics of each of the micromodules. The statistics include one or any combination of number of occurrences, average duration, or sequences of intermediate and end states (final assessments of micromodules). The module history component 90 computes (e.g., the processor 72 executes the component 90) the statistics based on user available data (e.g., from wearable device 12 (FIG. 2), electronics device 14 (FIG. 3), network storage, etc.) and/or peers data (e.g., collected from social media sites, etc.).

The human model component 88 comprises models of the user that are built based on available data (e.g. app usage, sensor data), available information related to preference and constraints of the users, available information for peers (e.g. same gender, race, age, lifestyle). For instance, the human model component 88 (or a data structure in coupled storage) may access user and/or peer data from data structures 96 coupled to the I/O devices 74 (directly or via network storage) or the data bus 78 (e.g., via storage device), the data structures 96 configured to store user profile data including the real-time measurements of parameters for a large population of users, personal information of the large population of users, previously generated statements related to the large population of users, etc. In some embodiments, the data structures 96 are configured to store health-related information of the user. The user profile data is organized to enable the human model component 88 to model various aspects of a user in a way that supports simple querying as well as complicated data analysis. The data structures 96 may be a backend database of the computing system 20. In some embodiments, however, the data structures 96 may be in the form of network storage and/or cloud storage directly connected to the network 18 (FIG. 1). In some embodiments, the data structures 96 may serve as backend storage of the computing system 20 as well as network storage and/or cloud storage. The data structures 96 are updated periodically, aperiodically, and/or in response to a request from the wearable device 12, the electronics device 14, and/or the operations of the computing system 20.

The scheduling component 92 manages the micromodules over time (e.g. not more than one micromodule at the time—if at time, t, an opportunity with high score is detected and another micromodule is ongoing, the scheduling component 92 defines which priorities should be followed). This management depends on the settings of the application. For instance, if a maximum of M chains have settled at a given moment in time, and a new chain/opportunity comprises a high score, the scheduling component 92 defines the M chains to be proposed to the user at that moment in time. Note that there may be plural micromodules, each having different targets, running simultaneously. For instance, one micromodule may correspond to nutritional habits, whereas another micromodule corresponds to physical activity. The scheduling component 92 uses the data from the module history component 90 and the human model component 88 to score, over time, ongoing chains or micromodules and, eventually, new opportunities.

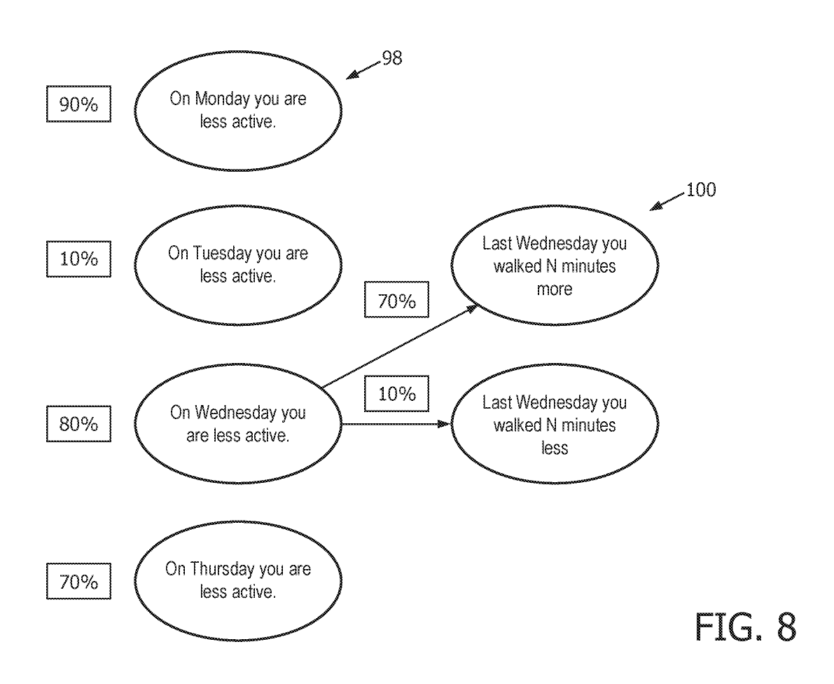

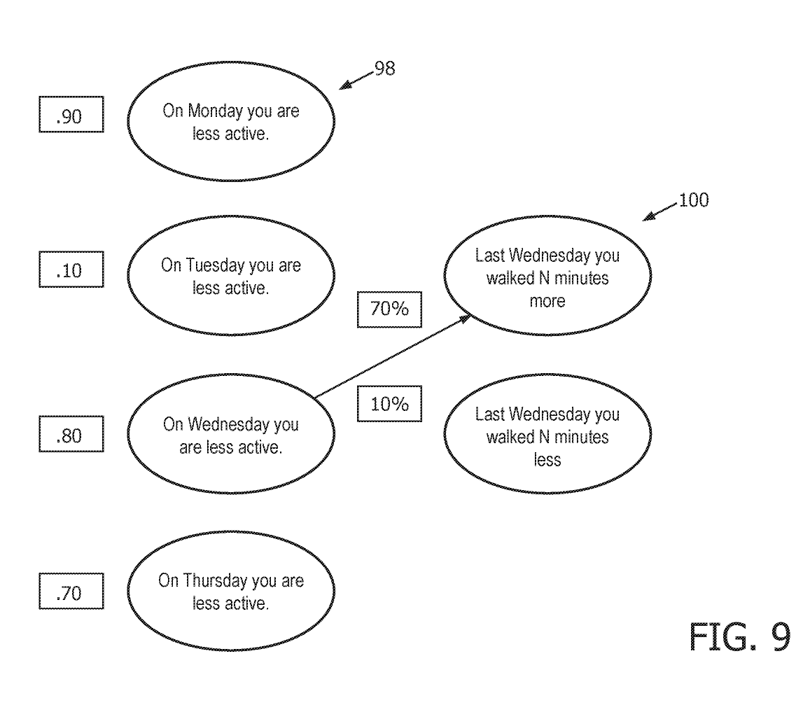

The parametrization component 94 adapts the proposed opportunity at time, t, based on learned parameter(s) over time from the user or peer responses to previous micromodule, which enables adjustment of a target of each micromodule. For instance, the follow-up of an opportunity (as described further in association with FIGS. 8-9) may be defined based on the learned parameters (e.g., a strategy of following up on a defined chain may be given preference to peer support or to follow up only if the user is tracking progress in the subsequent week). In some embodiments, the parametrization component 94 (and/or other components) may be omitted and its corresponding functionality combined with other modules. For instance, the parametrization functionality may be omitted, with the module orchestration unit 86 performing this functionality via the module history component 90.

The chain control logic 95 tracks the scoring of intermediate nodes in a chain, and is described further below.

The communication module formats the notifications to be issued according to one or any combination of human-perceivable format (e.g., visually, audibly, using tactile feedback, including Braille, etc.). In one embodiment, the communication module may comprise card presentation functionality. As used herein, content cards generated for a specific parameter define a “family” of statements associated with the specific parameter. For example, the content cards generated for sleep quality define a family of statements related to sleep quality, while the content cards generated for running define a family of statements related to running. The content cards may be configured to present one statement per card, though in some embodiments, additional statements may be presented. Different families may define different numbers of statements for presentation. In some embodiments, the content cards may be configured to present respective statements related to the feedback of an activity performance. In some embodiments, the content cards may be configured to present statements comprising educational information. In some embodiments, the content cards may be configured to present respective statements comprising insightful analysis of the user's health-related conditions. In some embodiments, the content cards may comprise only text statements. In some embodiments, the content cards may comprise content in multiple formats including but not limited to text, audio, video, flash, hyperlink to other sources, etc. It should be appreciated that the content cards may be generated for purposes other than the examples described above, and the format of the content cards may be adjustable for presentation on different user devices. The examples set forth above are for illustrative purposes; and the present disclosure is not intended to be limiting. For instance, presentation of the notifications is not limited to content card formats.

In one embodiment, the communications module is configured to receive the statements associated with each node and configure into content card format and present the respective content cards to the user. The communications module may prepare the presentation of the content cards based on the settings pre-defined by the user and/or the configuration of each individual user device. The settings pre-defined by the user may comprise how the user wants to be notified with the content cards, for example, in a text format, in a chart format, in an audio format with low-tone female voice, in a video/flash format, and/or the combinations thereof. The settings pre-defined by the user may further comprise when and how often the user wants to be notified with the content cards, for example, every evening around 9:00 pm, every afternoon after exercise, every week, every month, in real-time, and/or the combination thereof. The settings pre-defined by the user may further comprise a preferred user device to receive the content card if the user has multiple devices. The configuration of each individual user device may include the size and resolution of the display screen of a user device, the caching space of the user device, etc. In some embodiments, the communications module may determine the connection status of the user device before sending the content cards. If the user device is determined unavailable due to power off, offline, damaged, etc., the communications module may store the generated content card in memory 76 and/or upload the generated content card to the data structure 96. Once the user is detected logged-in using one of his or her user devices, the generated content card is transmitted to the user device for presentation. In some embodiments, if the preferred user device is unavailable, the communications module adjusts the content card for presentation in the logged-in user device.

In some embodiments, the communications module may convert a statement to one or more variations of the statement so that the converted statement matches a desired tone of voice, target population, or language, etc. The variations of a word and/or a statement may be acquired from a linguistic knowledge base. For example, the statement “Your sleep quality is highest after Mondays” may be converted to “You sleep well after Mondays.”

In some embodiments, the communications module may generate a large number of visual representations of a human body. The measurement data based on body sensors may be used to determine one or more images. The one or more images are further included in the content card or cards for presentation. Therefore, each content card presents a health picture of the individual, which can also be forwarded to a caregiver for reference. In some embodiments, the content card or cards may be presented in an orchestral arrangement of a melody played back to the user. It should be appreciated that the examples of card presentation described above are for illustrative purpose. The present disclosure is not intended to be limiting. In some embodiments, the communications module may supplement additional information to the statements for presentation of the content card. The additional information comprises professional advices on how to improve the user's health condition, feedbacks from a community environment, educational resources, etc.

The communications module further enables communications among network-connected devices and provides web and/or cloud services, among other software such as via one or more APIs. For instance, the communications module may receive (via I/O interfaces 74) input data (e.g., a content feed) from the wearable device 12 and/or the electronics device 14 that includes sensed data and a context for the sensed data, data from third-party databases (e.g., medical data base), data from social media, data from questionnaires, data from external devices (e.g., weight scales, environmental sensors, etc.), among other data. The content feed may be continual, intermittent, and/or scheduled. The communications module operates in conjunction with the I/O interfaces 74 to provide the notifications to the wearable device 12 and/or the electronics device 14.

Execution of the application software 30B (and associated components 86-94 and the communications module) may be implemented by the processor 72 under the management and/or control of the operating system. The processor 72 may be embodied as a custom-made or commercially available processor, a central processing unit (CPU) or an auxiliary processor among several processors, a semiconductor based microprocessor (in the form of a microchip), a macroprocessor, one or more application specific integrated circuits (ASICs), a plurality of suitably configured digital logic gates, and/or other well-known electrical configurations comprising discrete elements both individually and in various combinations to coordinate the overall operation of the computing system 20.

The I/O interfaces 74 comprise hardware and/or software to provide one or more interfaces to the Internet 18, as well as to other devices such as a user interface (UI) (e.g., keyboard, mouse, microphone, display screen, etc.) and/or the data structures 82,84, 96. The user interfaces may include a keyboard, mouse, microphone, immersive head set, display screen, etc., which enable input and/or output by an administrator or other user. The I/O interfaces 74 may comprise any number of interfaces for the input and output of signals (e.g., analog or digital data) for conveyance of information (e.g., data) over various networks and according to various protocols and/or standards. The user interface (UI) is configured to provide an interface between an administrator or content author and the computing system 20. The administrator may input a request via the user interface, for instance, to manage the template database 82. Upon receiving the request, the processor 72 instructs a template building component to process the request and provide information to enable the administrator to create, modify, and/or delete the templates. As indicated above, the content author may use the user interface to label statements and establish plural possible pathways among the starting, intermediate, and target nodes.

When certain embodiments of the computing system 20 are implemented at least in part with software (including firmware), as depicted in FIG. 4, it should be noted that the software (e.g., application software 30B, associated components 86-94 and the communications module) can be stored on a variety of non-transitory computer-readable medium for use by, or in connection with, a variety of computer-related systems or methods. In the context of this document, a computer-readable medium may comprise an electronic, magnetic, optical, or other physical device or apparatus that may contain or store a computer program (e.g., executable code or instructions) for use by or in connection with a computer-related system or method. The software may be embedded in a variety of computer-readable mediums for use by, or in connection with, an instruction execution system, apparatus, or device, such as a computer-based system, processor-containing system, or other system that can fetch the instructions from the instruction execution system, apparatus, or device and execute the instructions.

When certain embodiments of the computing system 20 are implemented at least in part with hardware, such functionality may be implemented with any or a combination of the following technologies, which are all well-known in the art: a discrete logic circuit(s) having logic gates for implementing logic functions upon data signals, an application specific integrated circuit (ASIC) having appropriate combinational logic gates, a programmable gate array(s) (PGA), a field programmable gate array (FPGA), relays, contactors, etc.

Having described the underlying hardware and software of certain embodiments of a micromodule management system, some additional information on the formation of micromodules is described hereinafter. For instance, it is assumed, for purposes of brevity, that the statements are already constructed and hence accessed from the notifications data structures 84. In general, a statement generating component comprises algorithms that instruct a processor to generate one or more statements pertaining to a parameter. In some embodiments, the statements may be ranked for each parameter by the ranking component implementing a truth engine. In some embodiments, ranking may not be implemented. The statements are stored in the notifications data structures 84 or in other storage and accessed by a content author for use in conjunction with a chain building component. A node association component is used by the content author to associate the predetermined data structures (e.g., notifications or statements) provided by a data structure definition component (and stored in the notifications data structures 84) with either starting nodes, intermediate nodes, or target nodes.

For instance, the content author may be presented via the node association component a graphical user interface (or other interface of a software tool) that enables the content author to label the statements based on their content as start- or end-points (e.g., starting nodes and target nodes, respectively) of narratives. In one embodiment, start-point content refers to an opportunity, e.g., “On Saturdays after work you are typically less active than other evenings. Can you consider becoming more active then?”, and an end-point could be, for example, a positive observation or assessment related to a measure “Your average walking distance in Saturday evenings is 50% more than other work days. Well done! This really helps you to reach your targets earlier.” The content author further labels the statements he or she deems as appropriate as intermediate statements. The intermediate nodes comprise intermediate data structures (e.g., notifications or statements) that comprise a network of all possible intermediate statements that join the start points with the end points. Collectively, the plurality of intermediate nodes comprises a network of intermediate nodes.

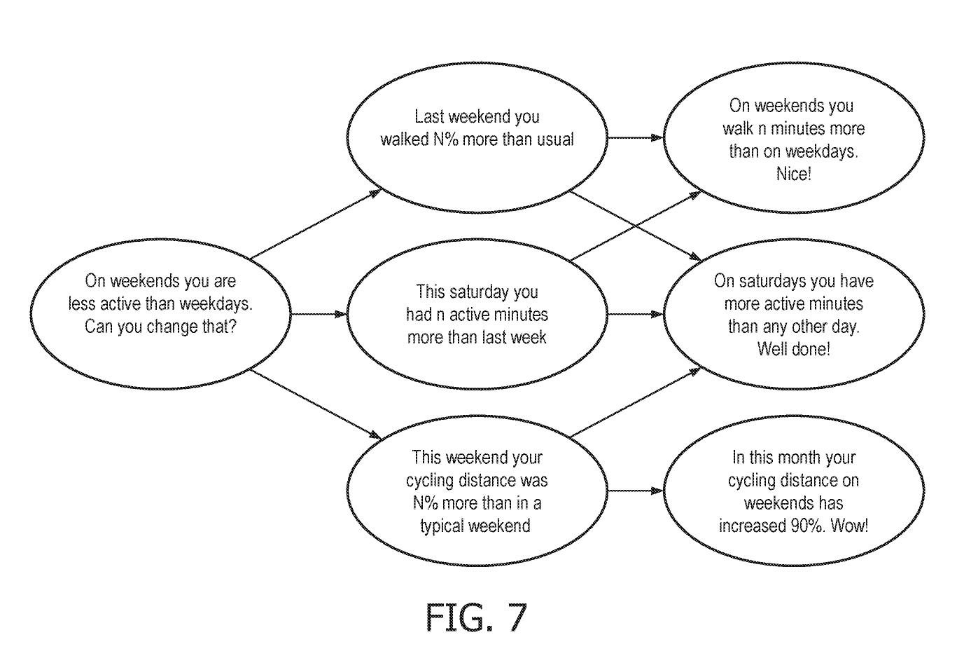

A pathway establishment component is another software tool used by the content author to establish plural (e.g., all) possible pathways from the starting nodes to the target nodes (e.g., end points). A path or pathway refers to a narrative, which is a chain of statements that leads from a start-point (e.g., starting node) to an end-point (e.g., target node), collectively creating a micromodule. Using the pathway establishment component (e.g., another interactive GUI), the content author identifies all possible paths in the collection of statements, and populates a statement table for each of the starting and ending nodes based on the possible paths. In other words, each starting and ending node has a statement table that lists all possible next nodes (next statements) along with plural parameters for each statement. In one embodiment, there are typically several possible paths from a start-point to an end-point and there are also multiple paths to different end-points from one start-point. An intermediate statement in the path example introduced above could be for example, “This Saturday you have been more active than on typical Saturday evenings”. An example of different paths or pathways is illustrated in FIG. 7, described further below. The chain building component receives input data over a certain interval of time. The interval of time may be user configured or pre-programmed. During the monitoring of a user (e.g., receiving input data from the wearable device 12 and/or the electronics device 14) and/or at predetermined intervals or after certain events (e.g., after determining an opportunity for positive behavioral change), a measures compute component computes measures or scores for the starting point statements (starting nodes) based on the input data. Afterwards, the chains are progressively built based on received input data, which includes user data and contexts associated with the data, with each node (e.g., comprising the next statement) selected after an intervening period of time where data is gathered according to the goal the health program seeks to achieve for the user.

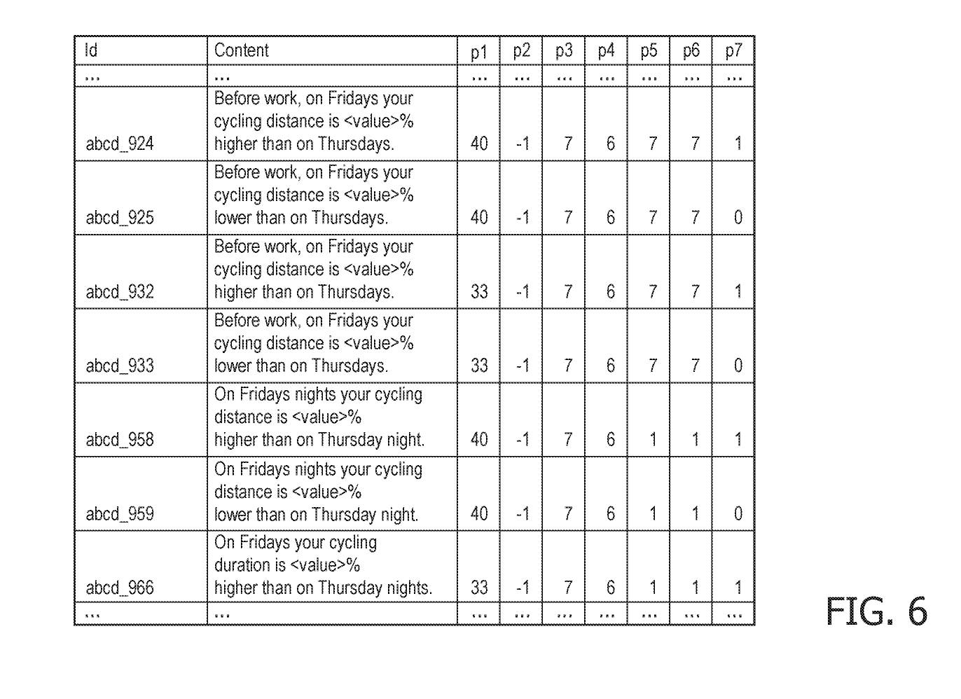

Referring to FIG. 6, the generation of the micromodule may be performed manually or automatically based on the coding of the statements. For instance, an insight generator may be used to score insights/statements. Referring to the example list of statements in FIG. 6, each statement has a unique ID and parameters that describe the context, measurement, and direction of the statement. The scoring of a statement is based on the parameters and the data from the user. The statement scoring algorithm of the insight generator collects the statistics of the measurements represented by the parameters and computes a unique score for each statement. The value of the score is between 0 and 100.

Referring to FIG. 7, shown is one example of a set of connected insights/statements. The left-most insight is a start node and the nodes on the right are possible target nodes. The intermediate nodes are selected such that they represent intermediate short-term steps between the start node and the target nodes. The chains (or micromodules) are represented by lists of statement IDs. A large collection of statement candidates is generated, which address all detectable contexts, measurements and their statistical relations. The statements are labelled based on their content as start- or end-points of narratives. A path of a narrative is a sequence of statements that leads from a start-point to an end-point. All possible paths are identified in the collection of statements. There are typically several possible paths from a start-point to an end-point, and there are also multiple paths to different end-points from one start-point. An intermediate statement in the path introduced above could be for example, “This Saturday you have been more active than on typical Saturday evenings”. In one embodiment, a chain entry point is detected based on the insight generator scoring. For example, the start of a chain is triggered if the confidence score is, for example, higher than 80%. In some embodiments, the user is informed about the detection of an opportunity in a dashboard (e.g., GUI presented to a user on his/her user device), and asked if he/she wants to start a personal program module that helps to improve the lifestyle in the referred context. When a chain is triggered, the chain control logic 95 start tracking the scoring of the intermediate nodes in the chain. When one of the intermediate nodes scores higher than others, and above a certain threshold, the card is published (e.g., communicated to, and presented by a user device) to the user and the chain control logic 95 moves to the next node and starts tracking the scores of the subsequent statements.

The process of the chain control logic 95 terminates when the path tracking ends in one of the target nodes. The path tracking may also be terminated, for example, if the path tracking is not proceeding from one node after a predetermined threshold period (e.g., within one month). A micromodule may be identified and stored by the micromodule management system based on its context (e.g. on mornings in the weekend) and measurements that the micromodule targets to improve (e.g., walking). In one embodiment, the module history component 90 (FIG. 4) records, per each type of micromodule, the statistics for the specific user and/or peer users, the statistics including number of occurrences, average duration, sequences of end states/assessments, transition probability between start and end states, etc. The aforementioned data allows certain embodiments of a micromodule management system to evaluate each chain, at a given moment in time, based on at least historical data.

To conceptually illustrate example operations of an embodiment of a micromodule management system (e.g., module orchestration unit 86, FIG. 4), attention is directed to FIGS. 8-9. Referring first to FIG. 8, at a given moment in time t, multiple micromodules may score higher than 80%. In this example, the micromodule management system can weight the several opportunities based on historical data. In the example depicted in FIG. 8, different start nodes (opportunities) 98 are represented on the left in FIG. 8, with each of the nodes 98 referring to different contexts of the lifestyle of the user (e.g. days of the week, including Monday, Tuesday, etc.). Over time, the micromodule management system learns, based on the historical data, which opportunities 98 are likely to be followed up by the user (as indicated by the percentage of follow-up to the left of each of the nodes 98). For instance, in the depicted example, in only 10% of the cases over time, the user has followed up an opportunity 98 regarding his/her activity level on Tuesday, while the percentages for the other days are substantially higher (e.g., 70-90%). The reason for the lower percentage of follow-up on Tuesday may be due to, for instance, personal constraints of the user on Tuesday. Similarly, the micromodule management system may learn the history related to the intermediate nodes 100. In FIG. 8, the percentage of successful cases (e.g., positive target node achieved shown to the left of the intermediate nodes 100) is stored/depicted for a chain (micromodule) related to increasing physical activity on Wednesday (70%). In this case, if the week after the number of active minutes decreases, in only 10% of the cases for intermediate nodes 100 (e.g., pertaining to walking N minutes less) will the user achieve the goal.

In one embodiment, the history is included in the scoring as an additional weight. The weight is based on a probability that the defined opportunity/intermediate node will lead to an end state with an achieved or overachieved goal. If more than one start node is scored higher, and the start weights based on the history include the ones represented on the left of FIG. 8, the micromodule management system underlines immediately the difference between a concrete opportunity on Monday and on Tuesday for this specific user as follows:

P[follow up|start point increase activity on Monday]=0.8

P[follow up start point increase activity on Tuesday]=0.1

Referring to FIG. 9, shows are the starting nodes 98 and the intermediate nodes 100 as similarly presented in FIG. 8, yet with the percentage of follow-up and percentage success replaced by historical starting weights and historical intermediate weights, respectively. In this example, the score of the Tuesday opportunity 98 decreases given the relatively low historical weight (0.1) of this starting point. In the same way, the historical weight of the intermediate nodes 100 enables the micromodule management system to prioritize chains that the user is likely to achieve. Looking at the example of FIG. 9, and the chain related to the Wednesday opportunity, if the user started this chain but the first intermediate node is showing a decrease in performance (e.g., walking N minutes less), it is likely that the user will not achieve the target. The micromodule management system may prioritize different strategies of electronic interventions (e.g. issuing tips linked with the intermediate statements) or give more emphasis to other ongoing chains for which the user is likely to achieve the targeted goal. This mechanism may be adapted based on the goal of the proposed chain (e.g., if the program/service aims to propose only micromodules that can be overachieved, the winning score is based only on “exceed settled target”). In some embodiments, assessments (i.e., end states) may include user text input (e.g. a user inputted text, such as “not possible to realize in this context”). In some embodiments, information stored in the module history component 88 (FIG. 4) may be used in case of conflicts of decision between newly discovered opportunities and on going micromodules. For instance, a health program may have settled with a maximum of N micromodules going on concurrently. If at time t, a new opportunity is discovered, certain embodiments of a micromodule management system, using the scheduling component 92 (FIG. 4), may score each of ongoing opportunities to determine, based on the observed progress, if any of the micromodules should be stopped to propose the new discovered opportunity.

In one embodiment, the micromodule management system stores in the module history component 90 (FIG. 4), per each module, the probability of success (e.g., exceed or achieved target) based on the average daily progress (or other additional context data). In some embodiments, the micromodule management system learns each micromodule by training a Hidden Markov model. In other words, the micromodule management system learns the probability of a given output/end assessment given the previous state or states. Reinforcement learning may be used to learn the preferences of a user. In some embodiments, a sequence may be learned, for example, using any graphical model (e.g., conditional random fields (CRF) algorithm, recurrent neural networks (RNN), or memory networks, including long short-term memory (LSTM) network, or gated recurrent unit (GRU) networks, or other sequential learning methods known in the fields of time-series analysis or natural language processing. At time t, for each of the ongoing micromodules, the probability of the next node having an achieved or overachieved state may be estimated based on the observations (e.g., context data measurements). In this way, certain embodiments of the micromodule management system may automatically prioritize the different ongoing chains and compare them with new discovered opportunities. The scheduling component 92 (FIG. 4) may compute, periodically (e.g., each week, or aperiodically in some embodiments), the status of ongoing chains and any newly discovered chains. The scheduling component 92 may prioritize chains and also define the end of a chain in case the probability of success (e.g., where success may be defined by coaches or user input) is lower than a settled threshold. Additional input from the user (e.g., free text), if related to a specific micromodule, may be stored with the module history component 90 and may be used in the scoring (e.g., as observation data). The module history component 90, as mentioned above, may also include the number of samples on which the statistics are based on and if based on personal or of other users (e.g., peers). These two factors may be included to weight the score of each chain over time. For instance, personal data history and peer history may have different scoring factors based on number of samples. Both of the data history can be included in scoring the opportunity (e.g., two additive components in the score).

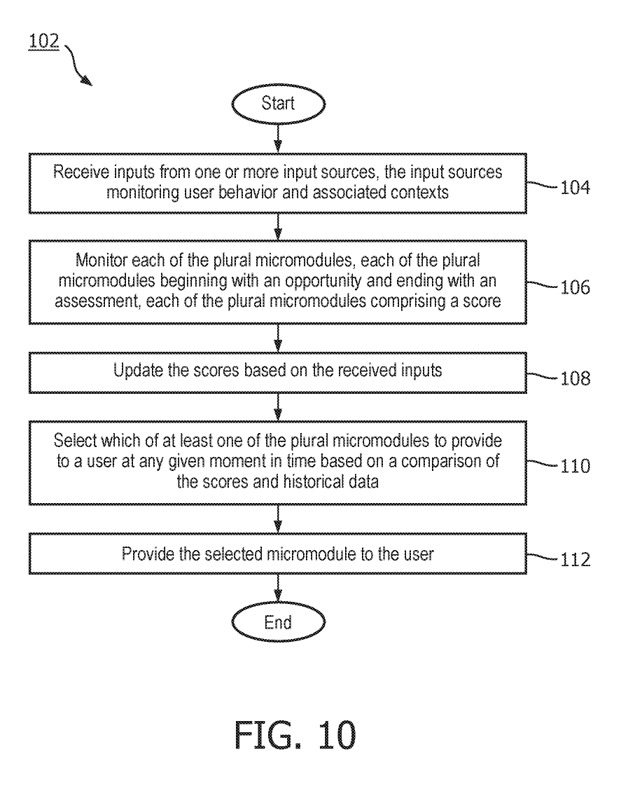

In view of the description above, it should be appreciated that one embodiment of a micromodule management method (e.g., implemented by the module orchestration unit 86, FIG. 4), depicted in FIG. 10 and referred to as a method 102 and encompassed between start and end designations, comprises receiving inputs from one or more input sources, the input sources monitoring user behavior and associated contexts (104); monitoring each of the plural micromodules, each of the plural micromodules beginning with an opportunity and ending with an assessment, each of the plural micromodules comprising a score (106); updating the scores based on the received inputs (108); selecting which of at least one of the plural micromodules to provide to a user at any given moment in time based on a comparison of the scores and historical data (110); and providing the selected micromodule to the user (112).

Any process descriptions or blocks in the flow diagram described above should be understood as representing modules, segments, or portions of code which include one or more executable instructions for implementing specific logical functions or steps in the process, and alternate implementations are included within the scope of an embodiment of the present invention in which functions may be executed substantially concurrently, and/or additional logical functions or steps may be added, depending on the functionality involved, as would be understood by those reasonably skilled in the art of the present invention.

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims. Note that various combinations of the disclosed embodiments may be used, and hence reference to an embodiment or one embodiment is not meant to exclude features from that embodiment from use with features from other embodiments. In the claims, the word “comprising” does not exclude other elements or steps, and the indefinite article “a” or “an” does not exclude a plurality. A single processor or other unit may fulfill the functions of several items recited in the claims. The mere fact that certain measures are recited in mutually different dependent claims does not indicate that a combination of these measures cannot be used to advantage. A computer program may be stored/distributed on a suitable medium, such as an optical medium or solid-state medium supplied together with or as part of other hardware, but may also be distributed in other forms.

At least the following is claimed:

1. A system for managing plural micromodules over time, each micromodule comprising plural nodes corresponding to electronic interventions that follow logically from one another to form a narrative or story, the system comprising:
a memory comprising instructions; and
one or more processors configured to execute the instructions to:
receive inputs from one or more input sources, the input sources monitoring user behavior and associated contexts;
monitor each of the plural micromodules, each of the plural micromodules beginning with an opportunity and ending with an assessment, each of the plural micromodules comprising a score;
update the scores based on the received inputs;
select which of at least one of the plural micromodules to provide to a user at any given moment in time based on a comparison of the scores and historical data; and
provide the selected micromodule to the user.

2. The system of claim 1, wherein the one or more processors are further configured to execute the instructions to identify each of the plural micromodules based on the context of its use and measurements that the micromodule targets to improve.

3. The system of claim 1, wherein the one or more processors are further configured to execute the instructions to monitor by recording, for each of the plural micromodules, statistics for a user or plural users.

4. The system of claim 3, wherein the statistics for each micromodule comprise one or any combination of number of occurrences, average duration, sequences of assessments, or a transition probability between the opportunity and the assessment.

5. The system of claim 1, wherein the one or more processors are further configured to execute the instructions to select by:
determining, for any given moment in time, an opportunity score for each opportunity of the respective plural micromodules based on the received inputs;
determining candidate opportunities by selecting the opportunities that meet or exceeds a threshold score;
first weighting the scores of the candidate opportunities based on the historical data, the historical data revealing which of the opportunities of the plural micromodules corresponding to the candidate opportunities are most likely to influence the user in reaching a goal; and
selecting the one of the candidate opportunities for the given moment in time based on the first weighted scores.

6. The system of claim 5, wherein the one or more processors are further configured to execute the instructions to select which of the at least one of the plural micromodules by:
at a second moment in time subsequent to the any given moment in time, determining node scores for each of the nodes associated with the selected one of the candidate opportunities based on the received inputs;
determining candidate nodes by selecting the nodes having scores that meet or exceeds a threshold score;
second weighting the scores of the candidate nodes based on the historical data, the historical data revealing which of candidate nodes corresponds to one of the plural micromodules most likely to influence the user in reaching the goal; and
selecting the one of the candidate nodes based on the second weighted scores.

7. The system of claim 6, wherein the one or more processors are further configured to execute the instructions to prioritize or adjust the plural micromodules corresponding to the candidate nodes based on the second weighted scores and updated behavior measurements of the user.

8. The system of claim 7, wherein the one or more processors are further configured to execute the instructions to prioritize by switching from the micromodules corresponding to the selected one of the candidate nodes to another of the candidate nodes based on an increased likelihood from the updated behavior measurements that the goal will not be reached.

9. The system of claim 7, wherein the one or more processors are further configured to execute the instructions to adjust by adjusting a strategy of electronic intervention issuance based on an increased likelihood from the updated behavior measurements that the goal will not be reached.

10. The system of claim 6, wherein the one or more processors are further configured to execute the instructions to receive a new opportunity, and based on the received new opportunity, update the scores of all of the micromodules, and determine whether one of the selected micromodules needs to be replaced with the new micromodule based on the updated scores.

11. The system of claim 1, wherein one or more of the assessments comprises user input, wherein the user input is included as part of the score.

12. The system of claim 1, wherein the one or more processors are further configured to execute the instructions to determine a probability of success in influencing the user to achieve the goal by assessing the probability for each of the plural micromodules on an ongoing basis based on the updates to the respective scores.

13. The system of claim 1, wherein the historical data includes number of samples for which statistics for a user or plural users for each of the plural micromodules are based, the statistics weighted by the historical data.

14. A computer-implemented method for managing plural micromodules over time, each micromodule comprising plural nodes corresponding to electronic interventions that follow logically from one another to form a narrative or story, the method comprising:
receiving inputs from one or more input sources, the input sources monitoring user behavior and associated contexts;
monitoring each of the plural micromodules, each of the plural micromodules beginning with an opportunity and ending with an assessment, each of the plural micromodules comprising a score;
updating the scores based on the received inputs;
selecting which of at least one of the plural micromodules to provide to a user at any given moment in time based on a comparison of the scores and historical data; and
providing the selected micromodule to the user.

15. The method of claim 14, further comprising identifying each of the plural micromodules based on the context of its use and measurements that the micromodule targets to improve.

16. The method of claim 14, wherein monitoring comprises recording, for each of the plural micromodules, statistics for a user or plural users, wherein the statistics for each micromodule comprise one or any combination of number of occurrences, average duration, sequences of assessments, or a transition probability between the opportunity and the assessment.

17. The method of claim 14, wherein selecting further comprises:
determining, for any given moment in time, an opportunity score for each opportunity of the respective plural micromodules based on the received inputs;
determining candidate opportunities by selecting the opportunities that meet or exceeds a threshold score;
first weighting the scores of the candidate opportunities based on the historical data, the historical data revealing which of the opportunities of the plural micromodules corresponding to the candidate opportunities are most likely to influence the user in reaching a goal;
selecting the one of the candidate opportunities for the given moment in time based on the first weighted scores;
at a second moment in time subsequent to the any given moment in time, determining node scores for each of the nodes associated with the selected one of the candidate opportunities based on the received inputs;
determining candidate nodes by selecting the nodes having scores that meet or exceeds a threshold score;
second weighting the scores of the candidate nodes based on the historical data, the historical data revealing which of candidate nodes corresponds to one of the plural micromodules most likely to influence the user in reaching the goal;
selecting the one of the candidate nodes based on the second weighted scores; and
prioritizing or adjusting the plural micromodules corresponding to the candidate nodes based on the second weighted scores and updated behavior measurements of the user, wherein prioritizing comprises switching from the micromodules corresponding to the selected one of the candidate nodes to another of the candidate nodes based on an increased likelihood from the updated behavior measurements that the goal will not be reached, and wherein adjusting comprises adjusting a strategy of electronic intervention issuance based on an increased likelihood from the updated behavior measurements that the goal will not be reached.

18. The method of claim 17, further comprising receiving a new opportunity, and based on the received new opportunity, updating the scores of all of the micromodules, and determining whether one of the selected micromodules needs to be replaced with the new micromodule based on the updated scores.

19. The method of claim 14, wherein a probability of success in influencing the user to achieve the goal is assessed for each of the plural micromodules on an ongoing basis based on the updates to the respective scores, and wherein the historical data includes number of samples for which statistics for a user or plural users for each of the plural micromodules are based, the statistics weighted by the historical data.

20. A non-transitory, computer readable medium encoded with instructions which, when executed by one or more processors, causes the one or more processors to:
receive inputs from one or more input sources, the input sources monitoring user behavior and associated contexts;
monitor each of the plural micromodules, each of the plural micromodules beginning with an opportunity and ending with an assessment, each of the plural micromodules comprising a score;
update the scores based on the received inputs;
select which of at least one of the plural micromodules to provide to a user at any given moment in time based on a comparison of the scores and historical data; and
provide the selected micromodule to the user.
